(19) United States
(12) Patent Application Publication
(10) Pub. No.: US 2010/0145115 A1
(43) Pub. Date: Jun. 10, 2010

(54) Method and Device for Formation and Transportation of Gas Hydrates in Hydrocarbon Gas and/or Condensate Pipelines

(75) Inventors: Are Lund, Trondheim (NO); Bernd Wittgens, Trondheim (NO); Paal Skjetne, Trondheim (NO)

Correspondence Address: CONLEY ROSE, P.C.;David A. Rose, P. O. BOX 3267, HOUSTON, TX, 77253-3267, US

(73) Assignee: SINVENT AS, Trondheim (NO)

(21) Appl. No.: 12/532,086

(22) PCT Filed: Mar. 17, 2008
(86) PCT No.: PCT/NO08/00104

§ 371 Date: Dec. 8, 2009

(30) Foreign Application Priority Data
Mar. 21, 2007 (NO) 2007 1495

(51) Int. Cl. C07C 9/00; C01B 6/00

(52) U.S. Cl. 585/15; 422/162

(57) ABSTRACT

The present invention relates to a method and system for treatment of a flow of fluid hydrocarbons containing water. The flow of hydrocarbons is introduced into a mixer (7) where it is mixed with liquid carbon dioxide (6) before the resulting mixture is cooled in a cooler (8) and conveyed to a reactor (9), in which all water present in the hydrocarbon flow will be in the form of hydrates, and said flow is conveyed to a pipeline (11) to be transported to its destination. The cooling system may comprise a choke or a mixer where the warm hydrocarbon flow is mixed with a cold flow of hydrocarbons.

The present invention relates to a method and system for transporting a fluid flow of hydrocarbons containing water. In the method said flow is transported through a treatment and transportation system including a pipeline.

The search and development for new gas and oil resources has for the last decades moved away from relative easily accessible continental waters, and towards deeper waters. This trend is visible in the Gulf of Mexico, Brazil, Barents Sea, Western Australia and North Sea. This development gives rise to several technological challenges.

Traditionally, in the North Sea, use of sub-sea templates and pipeline transport of the well-stream in multiphase pipelines has been restricted to a few tens of kilometres. Recently, better simulation and design tools resulted in improved equipment, which led to application of multiphase flow transport systems for transfer distances up to 110 km in the Gulf of Mexico.

The single most challenging problem for long gas transportation at deep water subsea, is the presence of natural gas hydrates. Natural gas hydrate is an ice-like compound consisting of light hydrocarbon molecules encapsulated in an otherwise unstable water crystal structure. These hydrates are formed at high pressures and low temperatures wherever a suitable gas and free water are present. These crystals can deposit on pipeline walls and in equipment, and in the worst case lead to complete plugging of the system. Costly and time-consuming procedures may be needed to restore flow again. In addition to the mere economic consequences, there are also numerous hazards connected with hydrate formation and removal, and there are known instances of pipeline ruptures and loss of human lives due to gas hydrates in pipelines. Although hydrate is generally thought of as a problem mostly for gas production, it is also a significant problem for condensate and oil production systems.

There are several available methods for dealing with hydrate problems. So far, the usual philosophy has been to take steps to avoid any hydrate formation at all. This can be achieved by keeping pressures low (often not possible from flow considerations), keeping temperature high (usually by insulating—which does not protect against shutdowns or long distances) removing the water completely (expensive equipment and difficult to carry out), or by adding chemicals that suppress hydrate formation thermodynamically. Insulation is very often used, but is not sufficient alone. Chemical addition of inhibitors or additives, especially mono ethylene glycol (MEG) or methanol (MeOH), is therefore the most widespread hydrate control mechanism in industries today. These antifreeze agents expand the pressure-temperature-area of safe operation, but are needed in large quantities. An addition in the order of up to 50 weight % of the total water fraction is common. The need for such large amounts places severe demands on logistics of transportation, storage capacity, injection, and require recollection facilities of additives (e.g. MEG). Further, the transport and injection processes for MeOH in particular, are also plagued with numerous leakages and spills.

Partly due to the huge amounts and large costs involved in using traditional inhibitors like MEG, there has over the last two decades been extensive effort devoted to finding chemicals which may be effective at controlling hydrates at much lower concentrations. Many companies and research institutes have contributed to this effort, where the result may be divided into three main categories: kinetic inhibitors, dispersants and modifiers. Kinetic inhibitors have an affinity for the crystal surface, and thereby can be used to prevent hydrate crystal growth. Dispersants act as emulsifiers, dispersing water as small droplets in the hydrocarbon liquid phase. This limits the possibilities for hydrate particles to grow large or to accumulate. The modifiers are to a certain extent a combination of the two other methods, attaching to the crystal surface, but also functioning as a dispersant in the liquid hydrocarbon phase. These methods have been somewhat successful but increase the cost of operation considerably. However, a significant problem seems to be that the best chemical additives so far produced have significant negative environmental effects.

There is growing understanding in the gas and oil industry that hydrate particles in a flow situation are not necessarily a problem per se. If the particles do not deposit on pipe walls or equipment and do not have a large impact on flow characteristics (i.e. their concentration is not too large), they simply flow with the rest of the fluids, without creating a problem situation. The challenge will therefore be to achieve this situation in a controlled manner, and making sure that hydrate formation does not take place randomly throughout the flow system.

In GB2358640 a method for transporting a flow of fluid hydrocarbons containing water through a treatment and transportation system including a pipeline is disclosed. In this method a hot flow of fluid hydrocarbon is introduced into a reactor (i.e. pipeline) where it is mixed with a cold flow of fluid hydrocarbons containing particles of gas hydrates. The effluent flow from the reactor may be further cooled in a heat exchanger (i.e. bare uninsulated pipeline) to ensure that all free water present therein is in the form of gas hydrates. The flow may then be split into one flow having a given content of gas hydrate particles recycled to the reactor while the rest is conveyed to a pipeline to be transported to its destination.

Another aspect which will definitely be affected by the present invention is corrosion in sub-sea pipelines. Considerable amounts of money and large resources in material and time are involved in protecting pipelines from corrosion, e.g. through conservation design (pipeline wall thickness i.e. steel quantity or quality) and through the use of corrosion inhibitors. Corrosion inhibitors are not necessarily used in the same amounts per pipeline as the hydrate inhibitors. Nevertheless because of the great number of pipelines in operation the total amount of chemicals is enormous. Corrosion inhibitors have usually a highly adverse effect on the environment. Much of the corrosion on pipeline and processing equipment is connected with free water, and successful results of the present invention may reduce this problem significantly.

The present invention provides a new method for pre-treatment of fluid hydrocarbons, mainly gas but not restricted to, containing water flowing through a treatment and transportation system including a pipeline. According to the invention the flow of fluid hydrocarbons is introduced into a mixer 7 where it is mixed with liquid carbon dioxide 6 from an injector, the resulting mixture is cooled to a temperature below the hydrate equilibrium temperature in a cooler 8, such as a choke, and then conveyed to a reactor, in which all water present in the hydrocarbon flow will be in the form of gas hydrates, and said flow is conveyed to a pipeline to be transported to its destination.

The present invention also provides a device for treating a flow of fluid hydrocarbons containing water, comprising in the flow direction, a hydrocarbon inlet 1, a mixer 7 with an inlet for liquid carbon dioxide 6, a cooler 8 and a reactor 9 where all water in the hydrocarbon flow will be converted to gas hydrates and a pipeline 11 for transporting the flow to its destination.

### FIGURES

FIG. 1 is a schematic drawing of the entire system.

FIG. 2 is a schematic drawing of an embodiment of the invention where the is cooler 8 is replaced with a mixer 12 where warm fluid hydrocarbon flow and liquid carbon dioxide from the mixer 7 is mixed with a cold fluid flow of hydrocarbons 13.

The flow of fluid hydrocarbons will normally come from a drilling hole well and will be relatively warm and under pressure. When the flow of fluid hydrocarbons is partly liquid oil or condensate it is generally preferred to cool the flow of hydrocarbons in a first cooler 4 before it is introduced into the above-mentioned mixer. If the temperature of the gas is sufficiently low the first cooler is not necessary.

It is sometimes desirable to add certain chemicals as corrosion inhibitors to the flow upstream to the reactor.

The method is particularly applicable in those cases where transportation takes place at a relative low temperature, both on land in a cool climate and at the sea bottom.

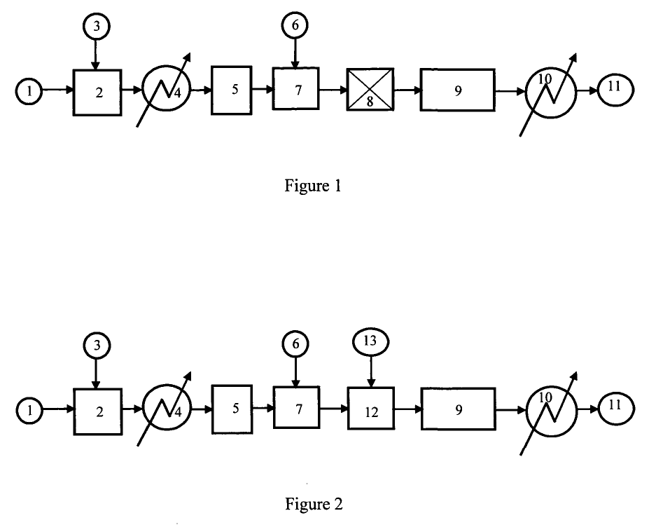

The system includes the following elements listed in the flow direction and connected with each other so that the hydrocarbons may pass through the entire system as illustrated in FIG. 1.
The system may comprise a connection to a hydrocarbon source containing water 1,
a first mixer 2 for hydrocarbon fluid and additive 3,
a first cooler 4,
a second mixer 5,
a third mixer 7 for hydrocarbon fluid and liquid carbon dioxide 6,
a second cooler 8
a reactor 9,
a third cooler 10, and
a pipeline 11.

The presented invention can be implemented in several configurations to adapt to varying hydrocarbon fluid conditions and compositions. When the flow of fluid hydrocarbons is partly liquid (oil, condensate, water), the system preferably includes a first cooler 4 and a mixer 5 upstream to the second cooler 8. The cooler 8 may be a part of the reactor 9.

The mixing 7 of hydrocarbon fluid and liquid carbon dioxide 6 may be accomplished by injection of liquid carbon dioxide into the fluid flow of hydrocarbons. An injector may be any kind of injector, but it may advantageously be of a type which distributes carbon dioxide liquid into many and small droplets with a large total droplet surface.

The inside of the system, in particular the inside of a first cooler 4, mixers 5 and 7, the cooler 8, the reactor 9, and the third cooler 10 may be coated with a water repellent material. Piping in between components may also advantageously be provided with such a coating material.

When the surroundings are rather cool, one or both of the coolers 4 and 10 used may be an uninsulated pipe. When the surroundings temperature is sufficiently low, this will provide satisfactory cooling without any further cooling medium.

In many cases it is advantageous to add different chemicals 3 to the flow of hydrocarbons, in particular during start-up and shut-down of liquid carbon dioxide supply and when changes are made in the operation. The system may accordingly contain a mixing mean 2 for such purpose and means of adding chemicals 3 to the flow.

In the following the present method and system will be described in more detail, again with reference to FIG. 1.

In the first embodiment warm hydrocarbon fluid flow containing water under pressure 1 are mixed with any desired chemicals 3 in a mixing means 2. If liquid water/oil/condensate is initially present in the fluid flow, most of the liquid may be separated out before mixing with chemicals. The chemicals in question may be any type of chemical used for transportation/storage of said fluid. The chemicals used should be acceptable for the environment and should generally be used preferably during start-up or shut-down of hydrocarbon fluid flow 1 and/or liquid carbon dioxide 6. During continuous operation, chemicals may even be left out completely.

The fluid from the mixer 2 may be precooled to a temperature above the hydrate equilibrium curve of the fluid (the melting curve of hydrate) in a cooler 4. At the bottom of the ocean said cooler may be an uninsulated tube, or it may be any type of cooler.

If the fluid flow from the cooler 4 contains liquid it is conveyed to a mixer 5 which may be any type of mixer. The mixer distributes the liquid in the fluid hydrocarbons as droplets. It should be noted that the mixer is not strictly necessary. The question whether or not a mixing operation is necessary depends on the characteristics of the fluid, i.e. the ability of the fluid to distribute the liquid as droplets in the fluid flow without any other influence than the turbulence which occurs when the fluid flows through a pipe. The mixer 5 may be a part of cooler 4 or mixer 7.

The fluid from the first cooler 4 or mixer 5 is conveyed to a mixer 7 which may be any type of mixer where it is mixed with liquid carbon dioxide from 6. In the mixer 7 liquid carbon dioxide is injected into the hydrocarbon fluid flow as small droplets by any type of injector. The diameter size of the carbon dioxide droplets is preferentially less than 1 mm. The liquid carbon dioxide 6 may alternatively be injected into the fluid flow of hydrocarbons at any point from cooler 4 to reactor 9. After mixer 7 the fluid flow is conveyed into a second cooler, such as a choke 8 where the fluid flow is cooled to a temperature below the hydrate equilibrium temperature of the hydrocarbon fluid flow, preferably to a temperature below 20° C. The fluid flow from the cooler 8 is conveyed into the reactor 9. The reactor 9 may be a pipeline.

The liquid carbon dioxide droplets in mixer 7, cooler 8, and reactor 9 will be below its partial vapour pressure and carbon dioxide gas will start to evaporate from the surface of the droplets. This process will cool down the carbon dioxide droplets, making them an ideal spot for hydrate nucleation (carbon dioxide hydrate) when contacted with free water in the fluid flow or preferentially with water condensing from the fluid flow by the temperature decrease in cooler 8. When this nucleation occurs on the surface of the carbon dioxide droplets, the droplets will be covered by a thin hydrate layer. However, heat from the surrounding fluid and the hydrate formation process (both on carbon dioxide droplets and elsewhere in the fluid mixture) in cooler 8 and reactor 9 will further evaporate carbon dioxide from the liquid carbon dioxide droplets. The internal pressure build-up in the hydrate covered carbon dioxide liquid droplets and/or flow turbulence will break up the hydrate layer on the surface. The hydrate layer will then be expelled into the fluid flow, and a new hydrate layer will form on the surface of the carbon dioxide liquid droplet. This will be repeated until the all liquid carbon dioxide is evaporated and/or dissolved in the fluid flow or all free water in the fluid flow is consumed by the process.

From the reactor 9 the fluid may be cooled down in a third cooler 10 to ambient temperature. At the bottom of the ocean said cooler may be an uninsulated pipe. The cooler 10 may also be any type of cooler and may be integrated as part of the reactor 9. After cooler 10 the fluid is conveyed at ambient temperature to a pipeline 11.

Dry hydrate particles formed on or at the carbon dioxide droplets or in the fluid flow will be stable if the temperature of the fluid flow in choke 8 and reactor 9 is below hydrate equilibrium temperature (sub-cooled) of the hydrocarbon fluid. Further water condensing because of decreasing/lower temperature in cooler 10 will moisten this dry hydrate and immediately be converted to hydrate (with hydrate forming gas components from the hydrocarbon fluid flow). New hydrate which is formed will accordingly increase the size of the hydrate particles, and also form new small hydrate particles when larger hydrate particles break up.

In a second embodiment (FIG. 2) of the invention the cooler 8 may be replaced with a mixer 12 where warm fluid hydrocarbon flow and liquid carbon dioxide from the mixer 7 is mixed with a cold fluid flow of hydrocarbons 13. In the mixer 12 the warm fluid flow from mixer 7 will be cooled to a temperature below the hydrate equilibrium temperature of the mixed hydrocarbon fluid flows, preferentially below 20° C. The fluid flow from the mixer 12 is conveyed into the reactor 9. The cold hydrocarbon fluid flow 13 may be from any upstream well(s) or recycled gas from any pump or compressor downstream pipeline 11.

In the mixer 7 liquid carbon dioxide is injected into the hydrocarbon fluid flow as small droplets by any type of injector. The diameter size of the carbon dioxide droplets is preferably less than 5 mm, in particular less than 1 mm. The liquid carbon dioxide 6 may alternatively be injected into the fluid flow of hydrocarbons in the mixer 12 or the reactor 9. The mixer 12 may be part of the reactor 9. The reactor 9 may be a pipeline.

The liquid carbon dioxide droplets in mixers 7 and 12 and reactor 9 will be below its partial vapour pressure and carbon dioxide gas will start to evaporate from the surface of the droplets. This process will cool down the carbon dioxide droplets, making them an ideal spot for hydrate nucleation (carbon dioxide hydrate) when contacted with free water in the fluid flow or preferentially with water condensing from the fluid flow by the temperature decrease in mixer 12. When this nucleation occurs on the surface of the carbon dioxide droplets, the droplets will be covered by a thin hydrate layer. However, heat from the surrounding fluid and the hydrate formation process (both on carbon dioxide droplets and elsewhere in the fluid mixture) in mixer 12 and reactor 9 will further evaporate carbon dioxide from the liquid carbon dioxide droplets. The internal pressure build-up in the hydrate covered carbon dioxide liquid droplets and/or flow turbulence will break up the hydrate layer on the surface. The hydrate layer will then be expelled into the fluid flow, and a new hydrate layer will form on the surface of the carbon dioxide liquid droplet. This will be repeated until the all liquid carbon dioxide is evaporated and/or dissolved in the fluid flow or all free water in the fluid flow is consumed by the process.

From the reactor 9 the fluid is cooled down in a third cooler 10 to ambient temperature. At the bottom of the ocean said cooler may be an uninsulated pipe. The cooler 10 may also be any type of cooler and may be integrated as part of the reactor 9. After cooler 10 the fluid is conveyed at ambient temperature to a pipeline 11.

Dry hydrate particles formed on or at the carbon dioxide droplets or in the fluid flow will be stable if the temperature of the fluid flow in mixture 12 and reactor 9 is below hydrate equilibrium temperature (sub-cooled) of the hydrocarbon fluid. Further water condensing because of decreasing/lower temperature in cooler 10 will moisten this dry hydrate and immediately be converted to hydrate (with hydrate forming gas components from the hydrocarbon fluid flow). New hydrate which is formed will accordingly increase the size of the hydrate particles, and also form new small hydrate particles when larger hydrate particles break up.

A further, general discussion of the present invention is given in the following.

Liquid carbon dioxide is in the near future expected to be a readily available product from carbon or hydrocarbon fuelled power plants and other large hydrocarbon fuel consumers, due to the fact that the carbon dioxide gas may not be allowed to escape in view of its global heating effect. A possible method for depositing the carbon dioxide is injection in hydrocarbon fields offshore, either in abandoned fields or for pressure support to enhance hydrocarbon recovery. This may give access to liquid carbon dioxide pipelines near or at new gas/condensate/oil fields for use in the present invention.

Hydrates formed at or near liquid carbon dioxide droplets will consist mainly of water and carbon dioxide. Provided the amount of liquid carbon dioxide 6 added to the hydrocarbon fluid flow is stoichiometric (about 1 kg liquid carbon dioxide to 10 kg water), most added carbon dioxide may be consumed in the hydrates formed. Thus, free and/or condensing water in the hydrocarbon fluid flow is converted to hydrates. With the water being in hydrate form, and the hydrate particles being dry (no excess water), it has been shown experimentally in flow loops with both model systems operating with real fluids and pressures and temperatures, that the resulting hydrate powder is easily transportable with the fluid flow. These tests also indicated that the particles will not aggregate or deposit on pipe walls or equipment. It is also a great advantage of the present invention that the absence of free water will reduce the risk of corrosion in pipelines and other installations.

The hydrate powder will not melt back to free water and carbon dioxide and/or natural gas until the temperature rises or the pressure becomes too low—which in reality will be at the end of the transport pipe. The hydrate powder may here be mechanically separated from the bulk fluid by a sieve, hydro cyclone, or any other suitable device, and melted in a separate device. Gas released from the hydrates will mainly be carbon dioxide which may be concentrated, compressed and reused in the present invention. Due to the availability of free heat at pipeline terminals for hydrate melting, the regeneration of carbon dioxide from hydrates is expected to be more economic than regeneration of MEG (mono ethylene glycol), also due to the smaller amounts needed for injection in a system (from 50 weight % for MEG to maximum 10 weight % for liquid carbon dioxide to weight free water).

The method given in the present invention may be applied with use of liquid propane or liquid butane or any other suitable liquid hydrate forming compound having the same basic properties as liquid carbon dioxide in a fluid flow of hydrocarbons.

The present invention can also be applied to any other processes were the removal of water (gaseous or liquid) from a fluid under high pressure is necessary.

1. Method for pretreatment of a flow of fluid hydrocarbons containing water flowing through a treatment and transportation system, the method comprising:
introducing the flow of fluid hydrocarbons into a first mixer;
mixing droplets of liquid carbon dioxide from an injector into the flow of fluid hydrocarbons, whereby a mixture of fluid hydrocarbons and liquid carbon dioxide is formed;
cooling the mixture of fluid hydrocarbons and liquid carbon dioxide in a first cooler to a temperature below the hydrate equilibrium temperature;
introducing the mixture of fluid hydrocarbons and liquid carbon dioxide into a reactor, wherein all water present therein is in the form of gas hydrates; and
conveying the mixture of fluid hydrocarbons and liquid carbon dioxide to a pipeline for transport to its destination.

2. The method of claim 1, wherein the cooler is a choke.

3. The method of claim 1, wherein the cooling of the mixture of fluid hydrocarbons and liquid carbon dioxide comprises mixing the flow of warm hydrocarbon fluid with a cold flow of hydrocarbons in a second mixer.

4. The method of claim 1, where the temperature of the mixture of fluid hydrocarbons and liquid carbon dioxide introduced into the reactor is below 20° C.

5. The method of claim 1, wherein the diameter size of the carbon dioxide droplets introduced into the first mixer is less than 5 mm.

6. The method of claim 1, wherein the fluid hydrocarbon flow comprises in part at least one of a liquid oil and a condensate.

7. The method of claim 1, further comprising adding corrosion inhibitors to the flow of fluid hydrocarbons upstream to the reactor.

8. The method of claim 1, wherein the liquid carbon dioxide is injected into the flow of fluid hydrocarbons at a point between a second cooler and the reactor.

9. Device for treating a flow of fluid hydrocarbons containing water, the devise comprising, in the flow direction, a hydrocarbon inlet, a mixer with an inlet for liquid carbon dioxide, a cooler, and a reactor, wherein all water in the flow of fluid hydrocarbon is converted to gas hydrates, and a pipeline for transporting the flow of fluid hydrocarbons to its destination.

10. The device of claim 9, wherein the cooler is one of a heat exchanger, a choke, or a mixer with an inlet for cold flow hydrocarbons.

11. The method of claim 3, wherein the temperature of the mixture of fluid hydrocarbons and liquid carbon dioxide introduced into the reactor is below 20° C.

12. The method of claim 1, wherein the diameter size of the carbon dioxide droplets introduced into the first mixer is less than 1 mm.

13. The method of claim 4, wherein the fluid hydrocarbon flow comprises in part at least one of a liquid oil and a condensate.

14. The method of claim 5, wherein the fluid hydrocarbon flow comprises in part at least one of a liquid oil and a condensate.
